SEQUENCE LISTING (15 sequences)

SEQ ID NO: 1 - DNA, 1194 bp, Pseudomonas putida
  source 1..1194: mol_type = genomic DNA; note = Pseudomonas putida KT2440
atgcgtcaaccagctcatctggcgctgacctgccttctggctctggctgctactctggctactccacttgcaccagctgctcaaggtcaggcagcagcaccgcttcacatccgtatcggttacctgggttaccgtccagctgctggtccactgctgtccaacatcattccagaaccaggtgatgcgggtttgcagggtgcagaactggctatcgttgactctaactctactggtcgtttcttgaaacagcagttccagctgacctctgcgaacgttgactctccagaagcactgttgcaggcagcacgtgcacagcacgaccagggtctgcgtgtgttcgttgttaacgcaccggcagcaagcctgcgtgctctgtccgcagctctgccggattctctgctgttcaacgcgggttctccggacgactctctgcgttctagccagtgcctgggtaacgtgctgcactctctgccgagccgtgcaatgctggcagatgcactggctcagtttctggttctgcgtaaatggcagcgtgctctgctggtagttggtcagactccggatgatcaggcatacgctgctgctttgcgtcgttccgttaaacgtttcggtgttcagctggttgcggagaaaacctggaccttcgacaacgatcagcgtcgttctgcacaggcagacatgccgctgttcactcagactgcggaatacgacgttgttctggtagcagacgaacgtggtgacttcggtgaatacctgccgtaccagacctggtatccgcgtccagtagcgggtactcagggtctgactccgactggttggcacaagaccgttgaaaccttcggtgctgcacaactccagaaacgtttcgaagctcaggctggtcgttggatgaacgaccgtgacttcgctgcttggatggcagttcgttctgttgcttctgctgcgtctaaactgcgtctggcagaaccacgtgctctccagcaactgctgctgtctgaacagctgccactggacggtttcaaaggtcgtaaactgagctaccgtccgtggaacggtgaactgcgtcagccgattccgctggttcagccgcgtgctcttgtttccacctctccgcaagaaggctttctgcatccgttcaacgaaatggactctctgggttacgacaaaccggaagttacctgctcttatccgtaa

SEQ ID NO: 2 - DNA, 990 bp, Pseudomonas putida
  source 1..990: mol_type = genomic DNA; note = Pseudomonas putida KT2440
atgcgtcgtccgttgttccaccgtgctctgctgtatccatctctgctgtctggtgctctggcgctggcatcttctcatactatggcagcaaccgcgtgggtttctaacgagaaagacaactctctgtctctgatcgacctgcaaactctggaagtgaccgaaaccttgccggttggtcagcgtccgcgtggtctgctgctgtctcatgactccaaactgctgtacatctgcgcttccgactctgatcgtgtgcaggttatggatgttgcaactcgtaagatcatcaaagaactgccgtctggtaaagatccggaacagttcgcactgcatccgaacgaccgttggctgtacgtttccaacgaagatgacgctctggttaccgttatcgataccgttaccgataaagtgctgggtcagatcgaagttggcatcgaaccagaaggtatggctgtatctccagacggtaagtgggctgtaaacacctctgaaaccaccaacatgctgcactggatcgacacttctactcagactctggcagattccactctggttgaccagcgtccacgtttcgtggagttctccaaagatggtagccgtctgtgggcttccgctgaaatcggtggcaccgttaccattctggatgttaccactcgtcaggttctgaaaactctgaacttccagatcaaaggtgtgcatccggacaaagttcagccggtaggtatcaaactgtctgctgacggcaaatacgcgttcgtagctctgggtccagcaaaccacgttgcggttatcgatgcaaagaccttcgacgttctggactacctgctggtgggtcgtcgtgtttggcagctggctttcactccagatcagtctcagcttctggcgaccaacggtgtttctggtgatgtttccgttatcgacgccaaacagctgaaagttctgaaatctgtcaaagttggtcgttacccgtggggtgttgttgttactccgtaa

SEQ ID NO: 3 - DNA, 741 bp, Pseudomonas putida
  source 1..741: mol_type = genomic DNA; note = Pseudomonas putida KT2440
atgaacgctctggacgtttctgacgttagcttcgcttacggtcagcgtgaagcgttgcgtcaggttggtttctctctggctccgggtcgtttcgctgctctgttgggtccaaacggtgctggtaaatccactctgatcgctctgctgactcgtctgtacgacctgcaacgtggtgacatccgtgtttgcggttgctctctgcgtaacgctgcacgtccagctcttcgtcagctgggtgttgtgttccagcagtctactctggacctggatctgtctgttgaacagaacctgcgttaccatgctgctctgcacggtctgtctcgtcgtcagtctcacctgcgtgttgaagctgaactggcacgtcagggtctgggtgaacgtcgtcgtgatcgtgttcgtgaactgaacatcggtcaccgtcgtcgcgttgaaatcgctcgtgcacttctgcacgaaccacgtttgctgctgctggatgaagcatctgtaggtctggacccagcaagtcgtctggctctgaaccagcatctgcgtcgtctgtgccgtgaacagcagctgtctgttctgtgggcaactcacctgctggacgaagttcagccgtctgacgacctgctgatcttgcaccagggtcgtctggttgcgtctggtacggcagacgctctgagcctggaacacggtggtgatctggatcacgctttcgcacgtctgaccaacgctccgtctggtgcgaacgcacgttaa

SEQ ID NO: 4 - DNA, 792 bp, Pseudomonas putida
  source 1..792: mol_type = genomic DNA; note = Pseudomonas putida KT2440
atgaacgcgtactggcagtgcttcaacggcatcatgctgcgtgaatggctgcgtttcgttctgcaacgtactcgtttgctgtctgcgctggttcgtccgctgctgtggctgctggtgttcgcagctggtttccgtgcagctctgggtatcgcgatcatcgaaccatacgacacctacatcccatacgaagtttacatcattccgggtctggcatgcatgatcttgctgttcaacggtatgcagggttctctgtctatggtttacgaccgtgaaatgggttctatgcgtgttctgctgacctctccgctgccacgtccgttcttgctgggttctaaactgctggctacctctctgatctctctgctgcaagtttacgcgttcttggcgatcgcttggctgtacggtgttcagccaccagcggcaggtctgctgatggctctgccggcactgctgctggttgcgttcatgctgtctgctctgggtctgctgctgtctaacgctatccgtcagctggagaacttcgcgggtgttatgaacttcgttatctttccgctgttctttctctcttctgcgctgtatccgctgtggaagatgcgtgaagcgtctcagtggctgtactggctgtgcgcggttaatccgttcactcacgcagttgaactggttcgtttcgcgctgtacgaacgtctgaacctgctggctctggcggtttgcctgggtctgactgcgctgttcactctgctggcgattctgaccttcaatccgcagcacgcggctctgcgtaaaccgcgttaa

SEQ ID NO: 5 - DNA, 3944 bp, Pseudomonas putida
  source 1..3944: mol_type = genomic DNA; note = Pseudomonas putida KT2440
atgcgtcaaccagctcatctggcgctgacctgccttctggctctggctgctactctggctactccacttgcaccagctgctcaaggtcaggcagcagcaccgcttcacatccgtatcggttacctgggttaccgtccagctgctggtccactgctgtccaacatcattccagaaccaggtgatgcgggtttgcagggtgcagaactggctatcgttgactctaactctactggtcgtttcttgaaacagcagttccagctgacctctgcgaacgttgactctccagaagcactgttgcaggcagcacgtgcacagcacgaccagggtctgcgtgtgttcgttgttaacgcaccggcagcaagcctgcgtgctctgtccgcagctctgccggattctctgctgttcaacgcgggttctccggacgactctctgcgttctagccagtgcctgggtaacgtgctgcactctctgccgagccgtgcaatgctggcagatgcactggctcagtttctggttctgcgtaaatggcagcgtgctctgctggtagttggtcagactccggatgatcaggcatacgctgctgctttgcgtcgttccgttaaacgtttcggtgttcagctggttgcggagaaaacctggaccttcgacaacgatcagcgtcgttctgcacaggcagacatgccgctgttcactcagactgcggaatacgacgttgttctggtagcagacgaacgtggtgacttcggtgaatacctgccgtaccagacctggtatccgcgtccagtagcgggtactcagggtctgactccgactggttggcacaagaccgttgaaaccttcggtgctgcacaactccagaaacgtttcgaagctcaggctggtcgttggatgaacgaccgtgacttcgctgcttggatggcagttcgttctgttgcttctgctgcgtctaaactgcgtctggcagaaccacgtgctctccagcaactgctgctgtctgaacagctgccactggacggtttcaaaggtcgtaaactgagctaccgtccgtggaacggtgaactgcgtcagccgattccgctggttcagccgcgtgctcttgtttccacctctccgcaagaaggctttctgcatccgttcaacgaaatggactctctgggttacgacaaaccggaagttacctgctcttatccgtaaatagttcccggccatgcacctctgtccatcgagaattcatcaggaagacattcaaatgaacgtaaacaataagggcagcgtctgtatttgcggcagcgaaatgctccctaaattcctcatttaccccatctgaggattgctttatgcgtcgtccgttgttccaccgtgctctgctgtatccatctctgctgtctggtgctctggcgctggcatcttctcatactatggcagcaaccgcgtgggtttctaacgagaaagacaactctctgtctctgatcgacctgcaaactctggaagtgaccgaaaccttgccggttggtcagcgtccgcgtggtctgctgctgtctcatgactccaaactgctgtacatctgcgcttccgactctgatcgtgtgcaggttatggatgttgcaactcgtaagatcatcaaagaactgccgtctggtaaagatccggaacagttcgcactgcatccgaacgaccgttggctgtacgtttccaacgaagatgacgctctggttaccgttatcgataccgttaccgataaagtgctgggtcagatcgaagttggcatcgaaccagaaggtatggctgtatctccagacggtaagtgggctgtaaacacctctgaaaccaccaacatgctgcactggatcgacacttctactcagactctggcagattccactctggttgaccagcgtccacgtttcgtggagttctccaaagatggtagccgtctgtgggcttccgctgaaatcggtggcaccgttaccattctggatgttaccactcgtcaggttctgaaaactctgaacttccagatcaaaggtgtgcatccggacaaagttcagccggtaggtatcaaactgtctgctgacggcaaatacgcgttcgtagctctgggtccagcaaaccacgttgcggttatcgatgcaaagaccttcgacgttctggactacctgctggtgggtcgtcgtgtttggcagctggctttcactccagatcagtctcagcttctggcgaccaacggtgtttctggtgatgtttccgttatcgacgccaaacagctgaaagttctgaaatctgtcaaagttggtcgttacccgtggggtgttgttgttactccgtaagaattttaacaggaggcaccccgggccctagagcgtaatcacccccattccatcttttttaggtgaaaacatgaacgctctggacgtttctgacgttagcttcgcttacggtcagcgtgaagcgttgcgtcaggttggtttctctctggctccgggtcgtttcgctgctctgttgggtccaaacggtgctggtaaatccactctgatcgctctgctgactcgtctgtacgacctgcaacgtggtgacatccgtgtttgcggttgctctctgcgtaacgctgcacgtccagctcttcgtcagctgggtgttgtgttccagcagtctactctggacctggatctgtctgttgaacagaacctgcgttaccatgctgctctgcacggtctgtctcgtcgtcagtctcacctgcgtgttgaagctgaactggcacgtcagggtctgggtgaacgtcgtcgtgatcgtgttcgtgaactgaacatcggtcaccgtcgtcgcgttgaaatcgctcgtgcacttctgcacgaaccacgtttgctgctgctggatgaagcatctgtaggtctggacccagcaagtcgtctggctctgaaccagcatctgcgtcgtctgtgccgtgaacagcagctgtctgttctgtgggcaactcacctgctggacgaagttcagccgtctgacgacctgctgatcttgcaccagggtcgtctggttgcgtctggtacggcagacgctctgagcctggaacacggtggtgatctggatcacgctttcgcacgtctgaccaacgctccgtctggtgcgaacgcacgttaacagacgagtcgaccatgaacgcgtactggcagtgcttcaacggcatcatgctgcgtgaatggctgcgtttcgttctgcaacgtactcgtttgctgtctgcgctggttcgtccgctgctgtggctgctggtgttcgcagctggtttccgtgcagctctgggtatcgcgatcatcgaaccatacgacacctacatcccatacgaagtttacatcattccgggtctggcatgcatgatcttgctgttcaacggtatgcagggttctctgtctatggtttacgaccgtgaaatgggttctatgcgtgttctgctgacctctccgctgccacgtccgttcttgctgggttctaaactgctggctacctctctgatctctctgctgcaagtttacgcgttcttggcgatcgcttggctgtacggtgttcagccaccagcggcaggtctgctgatggctctgccggcactgctgctggttgcgttcatgctgtctgctctgggtctgctgctgtctaacgctatccgtcagctggagaacttcgcgggtgttatgaacttcgttatctttccgctgttctttctctcttctgcgctgtatccgctgtggaagatgcgtgaagcgtctcagtggctgtactggctgtgcgcggttaatccgttcactcacgcagttgaactggttcgtttcgcgctgtacgaacgtctgaacctgctggctctggcggtttgcctgggtctgactgcgctgttcactctgctggcgattctgaccttcaatccgcagcacgcggctctgcgtaaaccgcgttaa

SEQ ID NO: 6 - DNA, 1491 bp, Ralstonia pseudosolanacearum
  source 1..1491: mol_type = genomic DNA
atggtagtccgtcgtaccgtgcttaaggcgattgcgggtacctctgttgccaccgtcttcgcgggaaagttaaccggcctgtctgctgtcgcagctgacgcagctccgctgcgtgtccgccgtaatcttcatggaatgaaaatggatgatcctgatctgagcgcgtatcgggagtttgtcggtatcatgaaaggtaaagatcagacccaggcgcttagctggttaggctttgccaatcagcatggcacgctgaatggcggatacaaatattgcccgcatggcgactggtattttctgccctggcatcgtggctttgtgctgatgtatgaacgtgctgtggcggcgttaacaggctataaaacctttgctatgccatattggaattggaccgaagatcgtctgttaccggaagcgtttactgcgaaaacctataatggcaagaccaatccgctgtacgtcccaaaccgtaatgaacttaccggcccatatgcactgactgacgcgatcgtcggccaaaaagaagtgatggataaaatctatgcggaaaccaactttgaagtatttggaacaagccggagcgtggatcggagtgtgcgtcccccactggtgcaaaatagcttagatccgaaatgggtgccgatgggcggtggaaatcagggcattctggaacgtaccccgcataacacagtgcacaataacattggtgcctttatgccaacagccgcctccccgcgtgatcccgtatttatgatgcaccacggcaacattgaccgtgtgtgggcaacctggaatgctctgggccgtaagaatagcacggatcctttgtggctgggcatgaagtttcctaataactacatcgacccccagggccgttactatacccagggtgtgagcgacctgctgagcaccgaggccttgggctatcgttacgatgtgatgcctcgtgcagataataaggtcgtcaataatgcgcgtgcggaacatctgctggcgctgtttaaaaccggggatagcgtgaaactggcagatcatatacgtttgcgtagcgttcttaagggcgagcatcccgttgcgacggctgtggaacccttgaatagcgcggtgcagtttgaagcgggcacggtgactggggcgttaggcgcggatgtggggaccggctcaaccaccgaagttgtggcactgatcaagaatatacgcataccgtataatgtgattagcattcgtgtatttgttaatctgccgaatgcgaatctggatgtgccagaaacagatccgcatttcgtgacatcgctgagctttctgacccatgcagctggacatgaccaccatgcgctgccgagcacgatggtaaatctgaccgataccctgaaagccctgaacattcgggatgacaatttttctataaatcttgtagcggtgccgcagcctggcgtggcggttgaatcgagcggaggcgtgaccccggaaagcattgaggtggcagtgatttaa

SEQ ID NO: 7 - protein, 397 aa, Pseudomonas putida
  source 1..397: note = Pseudomonas putida KT2440
MRQPAHLALTCLLALAATLATPLAPAAQGQAAAPLHIRIGYLGYRPAAGPLLSNIIPEPGDAGLQGAELAIVDSNSTGRFLKQQFQLTSANVDSPEALLQAARAQHDQGLRVFVVNAPAASLRALSAALPDSLLFNAGSPDDSLRSSQCLGNVLHSLPSRAMLADALAQFLVLRKWQRALLVVGQTPDDQAYAAALRRSVKRFGVQLVAEKTWTFDNDQRRSAQADMPLFTQTAEYDVVLVADERGDFGEYLPYQTWYPRPVAGTQGLTPTGWHKTVETFGAAQLQKRFEAQAGRWMNDRDFAAWMAVRSVASAASKLRLAEPRALQQLLLSEQLPLDGFKGRKLSYRPWNGELRQPIPLVQPRALVSTSPQEGFLHPFNEMDSLGYDKPEVTCSYP

SEQ ID NO: 8 - protein, 329 aa, Pseudomonas putida
  source 1..329: note = Pseudomonas putida KT2440
MRRPLFHRALLYPSLLSGALALASSHTMAATAWVSNEKDNSLSLIDLQTLEVTETLPVGQRPRGLLLSHDSKLLYICASDSDRVQVMDVATRKIIKELPSGKDPEQFALHPNDRWLYVSNEDDALVTVIDTVTDKVLGQIEVGIEPEGMAVSPDGKWAVNTSETTNMLHWIDTSTQTLADSTLVDQRPRFVEFSKDGSRLWASAEIGGTVTILDVTTRQVLKTLNFQIKGVHPDKVQPVGIKLSADGKYAFVALGPANHVAVIDAKTFDVLDYLLVGRRVWQLAFTPDQSQLLATNGVSGDVSVIDAKQLKVLKSVKVGRYPWGVVVTP

SEQ ID NO: 9 - protein, 246 aa, Pseudomonas putida
  source 1..246: note = Pseudomonas putida KT2440
MNALDVSDVSFAYGQREALRQVGFSLAPGRFAALLGPNGAGKSTLIALLTRLYDLQRGDIRVCGCSLRNAARPALRQLGVVFQQSTLDLDLSVEQNLRYHAALHGLSRRQSHLRVEAELARQGLGERRRDRVRELNIGHRRRVEIARALLHEPRLLLLDEASVGLDPASRLALNQHLRRLCREQQLSVLWATHLLDEVQPSDDLLILHQGRLVASGTADALSLEHGGDLDHAFARLTNAPSGANAR

SEQ ID NO: 10 - protein, 263 aa, Pseudomonas putida
  source 1..263: note = Pseudomonas putida KT2440
MNAYWQCFNGIMLREWLRFVLQRTRLLSALVRPLLWLLVFAAGFRAALGIAIIEPYDTYIPYEVYIIPGLACMILLFNGMQGSLSMVYDREMGSMRVLLTSPLPRPFLLGSKLLATSLISLLQVYAFLAIAWLYGVQPPAAGLLMALPALLLVAFMLSALGLLLSNAIRQLENFAGVMNFVIFPLFFLSSALYPLWKMREASQWLYWLCAVNPFTHAVELVRFALYERLNLLALAVCLGLTALFTLLAILTFNPQHAALRKPR

SEQ ID NO: 11 - protein, 496 aa, Ralstonia pseudosolanacearum
MVVRRTVLKAIAGTSVATVFAGKLTGLSAVAADAAPLRVRRNLHGMKMDDPDLSAYREFVGIMKGKDQTQALSWLGFANQHGTLNGGYKYCPHGDWYFLPWHRGFVLMYERAVAALTGYKTFAMPYWNWTEDRLLPEAFTAKTYNGKTNPLYVPNRNELTGPYALTDAIVGQKEVMDKIYAETNFEVFGTSRSVDRSVRPPLVQNSLDPKWVPMGGGNQGILERTPHNTVHNNIGAFMPTAASPRDPVFMMHHGNIDRVWATWNALGRKNSTDPLWLGMKFPNNYIDPQGRYYTQGVSDLLSTEALGYRYDVMPRADNKVVNNARAEHLLALFKTGDSVKLADHIRLRSVLKGEHPVATAVEPLNSAVQFEAGTVTGALGADVGTGSTTEVVALIKNIRIPYNVISIRVFVNLPNANLDVPETDPHFVTSLSFLTHAAGHDHHALPSTMVNLTDTLKALNIRDDNFSINLVAVPQPGVAVESSGGVTPESIEVAVI

SEQ ID NO: 12 - DNA, 30 bp, synthetic construct
  source 1..30: mol_type = other DNA
atgcgtcaaccagctcatctggcgctgacc

SEQ ID NO: 13 - DNA, 30 bp, synthetic construct
  source 1..30: mol_type = other DNA
ttaacgcggtttacgcagagccgcgtgctg

SEQ ID NO: 14 - DNA, 30 bp, synthetic construct
  source 1..30: mol_type = other DNA
atggtagtccgtcgtaccgtgcttaaggcg

SEQ ID NO: 15 - DNA, 30 bp, synthetic construct
  source 1..30: mol_type = other DNA
ttaaatcactgccacctcaatgctttccgg

(19) United States
(12) Patent Application Publication
(10) Pub. No.: US 2025/0215470 A1
(43) Pub. Date: Jul. 3, 2025

(54) GENETICALLY MODIFIED MICROORGANISM PRODUCING BLACK DYES AND METHOD FOR PRODUCING BLACK DYES

(71) Applicant: INDUSTRIAL TECHNOLOGY RESEARCH INSTITUTE, Hsinchu (TW)

(72) Inventors: Pei-Ching CHANG, Zhubei City (TW); Jhong-De LIN, Hsinchu City (TW); Hung-Yu LIAO, Taoyuan City (TW); Ya-Lin LIN, Hsinchu City (TW); Hsiang-Yuan CHU, Hsinchu City (TW)

(73) Assignee: INDUSTRIAL TECHNOLOGY RESEARCH INSTITUTE, Hsinchu (TW)

(21) Appl. No.: 18/975,464

(22) Filed: Dec. 10, 2024

(30) Foreign Application Priority Data
Dec. 29, 2023 (TW) 112151485

(51) Int. Cl. C12P 17/18; C07K 14/21; C12N 9/02; C12N 15/70

(52) U.S. Cl. CPC C12P 17/182; C07K 14/21; C12N 9/0071; C12N 15/70; C12Y 114/18001

(57) ABSTRACT

A genetically modified microorganism producing black dyes is provided. The genetically modified microorganism producing black dyes includes a first exogenous nucleic acid and a second exogenous nucleic acid. The first exogenous nucleic acid includes a nucleic acid encoding an ATP-binding cassette transporter (ABC transporter), wherein the nucleic acid encoding an ATP-binding cassette transporter includes a nucleic acid for ped gene cluster. The second exogenous nucleic acid includes a nucleic acid encoding tyrosinase.

### CROSS REFERENCE TO RELATED APPLICATIONS

This Application claims priority of Taiwan Patent Application No. 112151485, filed on Dec. 29, 2023, the entirety of which is incorporated by reference herein.

### REFERENCE TO AN ELECTRONIC SEQUENCE LISTING

The contents of the electronic sequence listing (0941-5467PUS1.xml; Size: 27,589 bytes; and Date of Creation: Feb. 10, 2025) is herein incorporated by reference in its entirety.

### TECHNICAL FIELD

The present disclosure relates to a genetically modified microorganism, a method of obtaining the same, and a method of using the same, and in particular, to a genetically modified microorganism that produces black dyes and a method of producing black dyes.

### BACKGROUND

Biosynthesis of chemicals is mainly achieved by microorganisms that pass raw materials through various enzymatic reactions. Nowadays, biosynthesis has become one of the main production methods to produce various chemicals.

How to further increase the production of biosynthetic chemicals is a major issue, and improving the growth ability and the efficiency of producing target chemicals of microorganisms is the core of biosynthetic chemicals.

The growth ability of microorganisms involves the culture environment and their own genes and protein expression, etc. It is known that rare earth ions are related to the growth and enzyme activity of microorganisms. However, there is still an urgent need for a modification method that enables microorganisms to effectively absorb and utilize rare earth ions to promote better growth efficiency of the microorganisms themselves, thereby simplifying the setting of various culture conditions and parameters and increasing the yield of target chemicals.

At present, black dyes containing benzidine have been strictly regulated according to the REACH regulations of the European Union, and thus the demand for biosynthesized black dyes is expected to increase. Melanin in living organisms has excellent anti-UV properties and is one of the main target chemicals for the biosynthesis of black dyes. Improving the biosynthetic efficiency of melanin is also the focus of current research.

### SUMMARY

An embodiment of the present invention provides a genetically modified microorganism producing black dyes. The genetically modified microorganism producing black dyes includes a first exogenous nucleic acid and a second exogenous nucleic acid. The first exogenous nucleic acid includes a nucleic acid encoding an ATP-binding cassette transporter (ABC transporter), wherein the nucleic acid encoding an ATP-binding cassette transporter includes a nucleic acid for ped gene cluster. The second exogenous nucleic acid includes a nucleic acid encoding tyrosinase.

In addition, an embodiment of the present invention provides a method for producing black dyes. The method for producing black dyes comprises culturing a genetically modified microorganism producing black dyes in the presence of a rare earth element and/or ion. The genetically modified microorganism producing black dyes includes a first exogenous nucleic acid and a second exogenous nucleic acid. The first exogenous nucleic acid includes a nucleic acid encoding an ATP-binding cassette transporter (ABC transporter), wherein the nucleic acid encoding an ATP-binding cassette transporter includes a nucleic acid for ped gene cluster. The second exogenous nucleic acid includes a nucleic acid encoding tyrosinase.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1A shows the map of plasmid V-RE1:

FIG. 1B shows the map of plasmid V-RE2;

FIG. 2 shows the standard curve of black dye concentration versus OD400.

FIG. 3 shows the effect of increasing the concentration of rare earth ions (lanthanum ions (La³⁺)) on the growth of the modified strain of Escherichia coli, ITRI-RB1;

FIG. 4 shows the improvement effect of different rare earth ions (lanthanum ions (La³⁺), cerium ions (Ce³⁺) and erbium ions (Er³⁺)) on the growth ability of the modified strain of Escherichia coli, ITRI-RB1;

FIG. 5 shows the rare earth ion (lanthanum ion (La³⁺)) uptake capabilities of Escherichia coli BL21 and the modified strain of Escherichia coli, ITRI-RB1:

FIG. 6 shows the effect of rare earth ions (lanthanum ions (La³⁺)) on the growth of the modified strains of Escherichia coli, ITRI-RB2 and ITRI-RB3; and

FIG. 7 shows the black dye production of the modified strains of Escherichia coli, ITRI-RB2 and ITRI-RB3.

### DETAILED DESCRIPTION

The following description is made for the purpose of illustrating the general principles of the invention and should not be taken in a limiting sense. The scope of the invention is best determined by reference to the appended claims.

The present disclosure may provide a genetically modified microorganism with enhanced rare earth ion absorption capacity. The rare earth ion mentioned above may be any rare earth ions, or any combination thereof, and has no particular limitation.

In one embodiment, the rare earth ion may comprise, but is not limited to, at least one of lanthanum (La) ion, cerium (Ce) ion, and erbium (Er) ion.

Compared to a source microorganism of the genetically modified microorganism with enhanced rare earth ion absorption capacity of the present disclosure, the rare earth ion absorption capacity of the genetically modified microorganism with enhanced rare earth ion absorption capacity of the present disclosure is raised and the growth rate thereof is increased.

The genetically modified microorganism with enhanced rare earth ion absorption capacity of the present disclosure can increase the absorption of rare earth ions, thereby increasing its growth rate, and thus compared to a source microorganism of the genetically modified microorganism with enhanced rare earth ion absorption capacity of the present disclosure, more microbial biomass can be obtained in the same culture time, or the original required culture time can be shortened.

Meanwhile, a source microorganism of the genetically modified microorganism with enhanced rare earth ion absorption capacity of the present disclosure mentioned above may comprise, but is not limited to a bacterium, an actinomycete, a yeast, a mold, etc.

In one embodiment, examples of the aforementioned bacterium that can be used as the source microorganism of the above-mentioned genetically modified microorganism with enhanced rare earth ion absorption capacity of the present disclosure may comprise a bacterium belonging to the genus Escherichia, a bacterium belonging to the genus Corynebacterium and a bacterium belonging to the genus Bacillus, but they are not limited thereto. Meanwhile, examples of a bacterium belonging to the genus Escherichia may comprise Escherichia coli, but they are not limited thereto. Examples of a bacterium belonging to the genus Corynebacterium may comprise, but are not limited to, Corynebacterium glutamicum. Examples of a bacterium belonging to the genus Bacillus may comprise Bacillus subtilis, but they are not limited thereto.

In another embodiment, the above-mentioned bacterium that can be used as the source microorganism of the above-mentioned genetically modified microorganism with enhanced rare earth ion absorption capacity of the present disclosure may comprise, but is not limited to, Escherichia coli, Corynebacterium glutamicum, Bacillus subtilis, etc. In one specific embodiment, the above-mentioned bacterium that can be used as the source microorganism of the above-mentioned genetically modified microorganism with enhanced rare earth ion absorption capacity of the present disclosure may be Escherichia coli. Furthermore, examples of Escherichia coli may comprise Escherichia coli BL21, BW25113, K12, DH5α, XL1-blue, but they are not limited thereto.

In one embodiment, examples of the aforementioned yeast that can be used as the source microorganism of the above-mentioned genetically modified microorganism with enhanced rare earth ion absorption capacity of the present disclosure may comprise, but are not limited to, a yeast belonging to the genus Yarrowia, a yeast belonging to the genus Saccharomyces and a yeast belonging to the genus Pichia. Examples of a yeast belonging to the genus Yarrowia may comprise Yarrowia lipolytica, but they are not limited thereto. Examples of a yeast belonging to the genus Saccharomyces may comprise, but are not limited to, Saccharomyces cerevisiae. Examples of a yeast belonging to the genus Pichia may comprise Pichia pastoris, but they are not limited thereto.

In another embodiment, the above-mentioned yeast that can be used as the source microorganism of the above-mentioned genetically modified microorganism with enhanced rare earth ion absorption capacity of the present disclosure may comprise, but is not limited to, Yarrowia lipolytica, Saccharomyces cerevisiae, Pichia pastoris, etc.

The disclosed genetically modified microorganism with enhanced rare earth ion absorption capacity may comprise, but is not limited to, a first exogenous nucleic acid, and the first exogenous nucleic acid mentioned above may comprise a nucleic acid encoding an ATP-binding cassette transporter (ABC transporter), but it is not limited thereto. The ATP-binding cassette transporter mentioned above may be a rare earth ion channel.

The nucleic acid encoding an ATP-binding cassette transporter mentioned above may comprise, but is not limited to, a nucleic acid for ped gene cluster.

In one embodiment, the above-mentioned ped gene cluster may comprise, but is not limited to, pedA1 gene, pedA2 gene, pedB gene and pedC gene. The pedA1 gene, pedA2 gene, pedB gene and pedC gene can respectively encode the PedA1 protein, PedA2 protein, PedB protein and PedC protein, and the PedA1 protein, PedA2 protein, PedB protein and PedC protein can constitute the above-mentioned ATP-binding cassette transporter.

In one embodiment, the sequence of the above-mentioned pedA1 gene contained in the above-mentioned ped gene cluster may comprise, but is not limited to, a sequence having more than 85% sequence identity with the sequence of SEQ ID NO. 1. In one specific embodiment, the sequence of the above-mentioned pedA1 gene contained in the above-mentioned ped gene cluster may comprise the sequence of SEQ ID NO. 1.

The term “having more than 85% sequence identity with” used in the present disclosure means that there is an identity of about 85%-100% between two sequences, such as about 85%, about 90%, about 95%, about 96%, about 97%, about 98%, about 99%, about 99.5%, about 99.8%, about 100%, but it is not limited thereto.

Moreover, in one embodiment, the sequence of the above-mentioned pedA2 gene contained in the above-mentioned ped gene cluster may comprise a sequence having more than 85% sequence identity with the sequence of SEQ ID NO. 2, but it is not limited thereto. In one specific embodiment, the sequence of the above-mentioned pedA2 gene contained in the above-mentioned ped gene cluster may comprise the sequence of SEQ ID NO. 2.

Furthermore, in one embodiment, the sequence of the above-mentioned pedB gene contained in the above-mentioned ped gene cluster may comprise, but is not limited to, a sequence having more than 85% sequence identity with the sequence of SEQ ID NO. 3. In one specific embodiment, the sequence of the above-mentioned pedB gene contained in the above-mentioned ped gene cluster may comprise the sequence of SEQ ID NO. 3.

In addition, in one embodiment, the sequence of the above-mentioned pedC gene contained in the above-mentioned ped gene cluster may comprise a sequence having more than 85% sequence identity with the sequence of SEQ ID NO. 4, but it is not limited thereto. In one specific embodiment, the sequence of the above-mentioned pedC gene contained in the above-mentioned ped gene cluster may comprise the sequence of SEQ ID NO. 4.

In another embodiment, the sequence of the nucleic acid encoding an ATP-binding cassette transporter mentioned above may comprise, but is not limited to, a sequence having more than 85% sequence identity with the sequence of SEQ ID NO. 5. In one specific embodiment, the sequence of the nucleic acid encoding an ATP-binding cassette transporter mentioned above may comprise the sequence of SEQ ID NO. 5.

In one embodiment, the sequence of the nucleic acid for ped gene cluster mentioned above may be derived from Pseudomonas putida, such as Pseudomonas putida KT2440, but it is not limited thereto.

In the foregoing embodiment in which the sequence of the nucleic acid for ped gene cluster mentioned above may be derived from Pseudomonas putida, for the pedA1 gene mentioned above, in one specific embodiment, the sequence thereof may comprise the sequence of SEQ ID NO. 1, but it is not limited thereto. Moreover, in the foregoing embodiment in which the sequence of the nucleic acid for ped gene cluster mentioned above may be derived from Pseudomonas putida, for the pedA2 gene mentioned above, in one specific embodiment, the sequence thereof may comprise, but it is not limited to, the sequence of SEQ ID NO. 2. Furthermore, in the foregoing embodiment in which the sequence of the nucleic acid for ped gene cluster mentioned above may be derived from Pseudomonas putida, for the pedB gene mentioned above, in one specific embodiment, the sequence thereof may comprise the sequence of SEQ ID NO. 3, but it is not limited thereto. In addition, in the foregoing embodiment in which the sequence of the nucleic acid for ped gene cluster mentioned above may be derived from Pseudomonas putida, for the pedC gene mentioned above, in one specific embodiment, the sequence thereof may comprise, but it is not limited to, the sequence of SEQ ID NO. 4.

Furthermore, in the foregoing embodiment in which the sequence of the nucleic acid for ped gene cluster mentioned above may be derived from Pseudomonas putida, in one specific embodiment, the sequence of the pedA1 gene mentioned above may comprise the sequence of SEQ ID NO. 1, the sequence of the pedA2 gene mentioned above may comprise the sequence of SEQ ID NO. 2, the sequence of the pedB gene mentioned above may comprise the sequence of SEQ ID NO. 3, and the sequence of the pedC gene mentioned above may comprise the sequence of SEQ ID NO. 4.

In addition, in the foregoing embodiment in which the sequence of the nucleic acid for ped gene cluster mentioned above may be derived from Pseudomonas putida, for one specific embodiment, the sequence of the nucleic acid encoding an ATP-binding cassette transporter mentioned above may comprise, but is not limited to, the sequence of SEQ ID NO. 5.

The location of the foregoing first exogenous nucleic acid contained in the genetically modified microorganism with enhanced rare earth ion absorption capacity of the present disclosure has no particular limitation, as long as the foregoing first exogenous nucleic acid can display its intended function, such as encoding a protein that conforms to the sequence information thereof. For example, the foregoing first exogenous nucleic acid contained in the genetically modified microorganism with enhanced rare earth ion absorption capacity of the present disclosure may be located in an expression vector or may be located in the genome of the genetically modified microorganism with enhanced rare earth ion absorption capacity of the present disclosure, but it is not limited thereto.

In one embodiment, the foregoing first exogenous nucleic acid contained in the genetically modified microorganism with enhanced rare earth ion absorption capacity of the present disclosure may be located in an expression vector. The expression vector mentioned above may comprise, but is not limited to, a plasmid, a cosmid, a viral vector, a chromosome (artificial chromosome), etc.

In one specific embodiment, the foregoing first exogenous nucleic acid contained in the genetically modified microorganism with enhanced rare earth ion absorption capacity of the present disclosure may exist in an expression vector, and the expression vector mentioned above is a plasmid.

Furthermore, the present disclosure also provides a culture method for improving the growth ability of microorganisms.

The culture method for improving the growth ability of microorganisms of the present disclosure mentioned above may comprise, but is not limited to, culturing a genetically modified microorganism with enhanced rare earth ion absorption capacity in the presence of a rare earth element and/or ion, and the foregoing genetically modified microorganism with enhanced rare earth ion absorption capacity may be any of the genetically modified microorganism with enhanced rare earth ion absorption capacity of the present disclosure mentioned above.

In the presence of the rare earth element and/or ion mentioned above, compared to a source microorganism of the foregoing genetically modified microorganism with enhanced rare earth ion absorption capacity, the foregoing genetically modified microorganism with enhanced rare earth ion absorption capacity may have better growth ability, and/or compared to in the absence of a rare earth element and/or ion, the foregoing genetically modified microorganism with enhanced rare earth ion absorption capacity may have better growth ability in the presence of a rare earth element and/or ion.

The rare earth element and/or ion mentioned above may be any rare earth elements and/or ions, or any combination thereof, and has no particular limitation. In one embodiment, the rare earth element mentioned above may comprise, but is not limited to, at least one of lanthanum, cerium, and erbium. In another embodiment, the rare earth ion mentioned above may comprise, but is not limited to, at least one of lanthanum ion, cerium ion, and erbium ion.

In one embodiment, for the culture method for improving the growth ability of microorganisms of the present disclosure, the rare earth element and/or ion mentioned above may be a rare earth ion, and the rare earth ion mentioned above may comprise, but is not limited to, at least one of lanthanum ion, cerium ion, and erbium ion.

For the culture method for improving the growth ability of microorganisms of the present disclosure, in one embodiment, the concentration of the rare earth element and/or ion mentioned above may be about 10-1000 mg/L, such as about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L, about 100 mg/L, about 125 mg/L, about 150 mg/L, about 200 mg/L, about 250 mg/L, about 300 mg/L, about 400 mg/L, about 500 mg/L, about 600 mg/L, about 700 mg/L, about 800 mg/L, about 900 mg/L, about 1000 mg/L, but it is not limited thereto. In one specific embodiment, for the culture method for improving the growth ability of microorganisms of the present disclosure, the rare earth element and/or ion mentioned above may be a rare earth ion, and the rare earth ion mentioned above may comprise, but is not limited to, at least one of lanthanum ion, cerium ion, and erbium ion, and the concentration of the rare earth ion mentioned above may be about 10-1000 mg/L, such as about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L, about 100 mg/L, about 125 mg/L, about 150 mg/L, about 200 mg/L, about 250 mg/L, about 300 mg/L, about 400 mg/L, about 500 mg/L, about 600 mg/L, about 700 mg/L, about 800 mg/L, about 900 mg/L, about 1000 mg/L, but it is not limited thereto.

In the culture method for improving the growth ability of microorganisms of the present disclosure, in one embodiment, the genetically modified microorganism with enhanced rare earth ion absorption capacity mentioned above may be cultured at about 25-40° C., such as about 25° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is not limited thereto.

Moreover, in the culture method for improving the growth ability of microorganisms of the present disclosure, in one embodiment, the genetically modified microorganism with enhanced rare earth ion absorption capacity mentioned above may be cultured at about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH 8.0, but it is not limited thereto.

Furthermore, in the culture method for improving the growth ability of microorganisms of the present disclosure, in one embodiment, the genetically modified microorganism with enhanced rare earth ion absorption capacity mentioned above may be cultured for about 4-24 hours, such as about 4 hours, about 5 hours, about 6 hours, about 7 hours, about 8 hours, about 9 hours, about 10 hours, about 11 hours, about 12 hours, about 15 hours, about 16 hours, about 18 hours, about 21 hours, about 24 hours, but it is not limited thereto.

Furthermore, in the culture method for improving the growth ability of microorganisms of the present disclosure, a culture medium used to culture the genetically modified microorganism with enhanced rare earth ion absorption capacity mentioned above has no particular limitation, as long as the genetically modified microorganism with enhanced rare earth ion absorption capacity mentioned above can grow therein. In one embodiment, in the culture method for improving the growth ability of microorganisms of the present disclosure, the culture medium used to culture the genetically modified microorganism with enhanced rare earth ion absorption capacity mentioned above may be Lysogeny broth (Luria Bertani broth) medium, a phosphate buffer (PB) with glucose, etc., but it is not limited thereto. In one specific embodiment, the culture medium used to culture the genetically modified microorganism with enhanced rare earth ion absorption capacity mentioned above may be Lysogeny broth medium. In another embodiment, the culture medium used to culture the genetically modified microorganism with enhanced rare earth ion absorption capacity mentioned above may be a phosphate buffer with about 5-15 g/L glucose, such as a phosphate buffer with about 10 g/L glucose.

In addition, the present disclosure may further provide a method for improving the growth ability of microorganisms, which may also be a method for preparing the genetically modified microorganism with enhanced rare earth ion absorption capacity of the present disclosure mentioned above.

The method for improving the growth ability of microorganisms of the present disclosure mentioned above may comprise introducing a first exogenous nucleic acid into a source microorganism to obtain a genetically modified microorganism, but it is not limited thereto, wherein compared to the source microorganism mentioned above, the genetically modified microorganism may have better growth ability.

With regard to the first exogenous nucleic acid used in the method for improving the growth ability of microorganisms of the present disclosure, it can be the same as the foregoing first exogenous nucleic acid in the genetically modified microorganism with enhanced rare earth ion absorption capacity of the present disclosure mentioned above, and thus all relevant descriptions thereof can be referred to all previous relevant descriptions regarding the first exogenous nucleic acid in the genetically modified microorganism with enhanced rare earth ion absorption capacity of the present disclosure mentioned above, and will not be repeated here.

Moreover, similarly, with regard to the source microorganism used in the method for improving the growth ability of microorganisms of the present disclosure, it can be the same as the source microorganism of the genetically modified microorganism with enhanced rare earth ion absorption capacity of the present disclosure mentioned above, and thus all relevant descriptions thereof can be referred to all previous relevant descriptions regarding the source microorganism of the genetically modified microorganism with enhanced rare earth ion absorption capacity of the present disclosure mentioned above, and will not be repeated here.

Furthermore, in the method for improving the growth ability of microorganisms of the present disclosure mentioned above, a manner for introducing the foregoing first exogenous nucleic acid into the foregoing source microorganism has no particular limitation, as long as the foregoing first exogenous nucleic acid can display its intended function, such as encoding a protein that conforms to the sequence information thereof. For example, the foregoing first exogenous nucleic acid can be introduced into an expression vector first, and then the expression vector can be introduced into the foregoing source microorganism, or the foregoing first exogenous nucleic acid can be directly introduced into the genome of the foregoing source microorganism, but it is not limited thereto.

In one embodiment, in the method for improving the growth ability of microorganisms of the present disclosure mentioned above, a manner for introducing the foregoing first exogenous nucleic acid into the foregoing source microorganism comprise introducing the foregoing first exogenous nucleic acid into an expression vector first, and then introducing the expression vector into the foregoing source microorganism. The expression vector mentioned above may comprise, but is not limited to, a plasmid, a cosmid, a viral vector, a chromosome (artificial chromosome), etc.

In one specific embodiment, in the method for improving the growth ability of microorganisms of the present disclosure mentioned above, a manner for introducing the foregoing first exogenous nucleic acid into the foregoing source microorganism comprise introducing the foregoing first exogenous nucleic acid into an expression vector first, and then introducing the expression vector into the foregoing source microorganism, and the expression vector mentioned above is a plasmid.

The present disclosure may further provide a genetically modified microorganism producing black dyes (melanin). The genetically modified microorganism producing black dyes of the present disclosure has an excellent black dye production capability.

Meanwhile, a source microorganism of the genetically modified microorganism producing black dyes of the present disclosure mentioned above may comprise, but is not limited to, a bacterium, an actinomycete, a yeast, a mold, etc.

In one embodiment, examples of the aforementioned bacterium that can be used as the source microorganism of the above-mentioned genetically modified microorganism producing black dyes of the present disclosure may comprise a bacterium belonging to the genus Escherichia, a bacterium belonging to the genus Corynebacterium and a bacterium belonging to the genus Bacillus, but they are not limited thereto. Meanwhile, examples of a bacterium belonging to the genus Escherichia may comprise Escherichia coli, but they are not limited thereto. Examples of a bacterium belonging to the genus Corynebacterium may comprise, but are not limited to, Corynebacterium glutamicum. Examples of a bacterium belonging to the genus Bacillus may comprise Bacillus subtilis, but they are not limited thereto.

In another embodiment, the above-mentioned bacterium that can be used as the source microorganism of the above-mentioned genetically modified microorganism producing black dyes of the present disclosure may comprise, but is not limited to, Escherichia coli, Corynebacterium glutamicum, Bacillus subtilis, etc. In one specific embodiment, the above-mentioned bacterium that can be used as the source microorganism of the above-mentioned genetically modified microorganism producing black dyes of the present disclosure may be Escherichia coli. Furthermore, examples of Escherichia coli may comprise Escherichia coli BL21, BW25113, K12, DH5α, XL1-blue, but they are not limited thereto.

In one embodiment, examples of the aforementioned yeast that can be used as the source microorganism of the above-mentioned genetically modified microorganism producing black dyes of the present disclosure may comprise, but are not limited to, a yeast belonging to the genus Yarrowia, a yeast belonging to the genus Saccharomyces and a yeast belonging to the genus Pichia. Examples of a yeast belonging to the genus Yarrowia may comprise Yarrowia lipolytica, but they are not limited thereto. Examples of a yeast belonging to the genus Saccharomyces may comprise, but are not limited to, Saccharomyces cerevisiae. Examples of a yeast belonging to the genus Pichia may comprise Pichia pastoris, but they are not limited thereto.

In another embodiment, the above-mentioned yeast that can be used as the source microorganism of the above-mentioned genetically modified microorganism producing black dyes of the present disclosure may comprise, but is not limited to, Yarrowia lipolytica, Saccharomyces cerevisiae, Pichia pastoris, etc.

Moreover, the genetically modified microorganism producing black dyes of the present disclosure may comprise, but is not limited to, a first exogenous nucleic acid and a second exogenous nucleic acid. The first exogenous nucleic acid mentioned above may comprise a nucleic acid encoding an ATP-binding cassette transporter (ABC transporter), but it is not limited thereto. The second exogenous nucleic acid mentioned above may comprise, but is not limited to, a nucleic acid encoding tyrosinase.

The nucleic acid encoding an ATP-binding cassette transporter mentioned above may comprise, but is not limited to, a nucleic acid for ped gene cluster.

In one embodiment, the ped gene cluster mentioned above may comprise, but is not limited to, pedA1 gene, pedA2 gene, pedB gene and pedC gene. The pedA1 gene, pedA2 gene, pedB gene and pedC gene can respectively encode the PedA1 protein, PedA2 protein, PedB protein and PedC protein, and the PedA1 protein, PedA2 protein, PedB protein and PedC protein can constitute the ATP-binding cassette transporter mentioned above.

In one embodiment, the sequence of the pedA1 gene mentioned above contained in the ped gene cluster mentioned above may comprise, but is not limited to, a sequence having more than 85% sequence identity with the sequence of SEQ ID NO. 1. In one specific embodiment, the sequence of the pedA1 gene mentioned above contained in the ped gene cluster mentioned above may comprise the sequence of SEQ ID NO. 1.

Moreover, in one embodiment, the sequence of the pedA2 gene mentioned above contained in the ped gene cluster mentioned above may comprise a sequence having more than 85% sequence identity with the sequence of SEQ ID NO. 2, but it is not limited thereto. In one specific embodiment, the sequence of the pedA2 gene mentioned above contained in the ped gene cluster mentioned above may comprise the sequence of SEQ ID NO. 2.

Furthermore, in one embodiment, the sequence of the pedB gene mentioned above contained in the ped gene cluster mentioned above may comprise, but is not limited to, a sequence having more than 85% sequence identity with the sequence of SEQ ID NO. 3. In one specific embodiment, the sequence of the pedB gene mentioned above contained in the ped gene cluster mentioned above may comprise the sequence of SEQ ID NO. 3.

In addition, in one embodiment, the sequence of the pedC gene mentioned above contained in the ped gene cluster mentioned above may comprise a sequence having more than 85% sequence identity with the sequence of SEQ ID NO. 4, but it is not limited thereto. In one specific embodiment, the sequence of the pedC gene mentioned above contained in the ped gene cluster mentioned above may comprise the sequence of SEQ ID NO. 4.

In another embodiment, the sequence of the nucleic acid encoding an ATP-binding cassette transporter mentioned above may comprise, but is not limited to, a sequence having more than 85% sequence identity with the sequence of SEQ ID NO. 5. In one specific embodiment, the sequence of the nucleic acid encoding an ATP-binding cassette transporter mentioned above may comprise the sequence of SEQ ID NO. 5.

In one embodiment, the sequence of the nucleic acid for ped gene cluster mentioned above may be derived from Pseudomonas putida, such as Pseudomonas putida KT2440, but it is not limited thereto.

In the foregoing embodiment in which the sequence of the nucleic acid for ped gene cluster mentioned above may be derived from Pseudomonas putida, for the pedA1 gene mentioned above, in one specific embodiment, the sequence thereof may comprise the sequence of SEQ ID NO. 1, but it is not limited thereto. Moreover, in the foregoing embodiment in which the sequence of the nucleic acid for ped gene cluster mentioned above may be derived from Pseudomonas putida, for the pedA2 gene mentioned above, in one specific embodiment, the sequence thereof may comprise, but it is not limited to, the sequence of SEQ ID NO. 2. Furthermore, in the foregoing embodiment in which the sequence of the nucleic acid for ped gene cluster mentioned above may be derived from Pseudomonas putida, for the pedB gene mentioned above, in one specific embodiment, the sequence thereof may comprise the sequence of SEQ ID NO. 3, but it is not limited thereto. In addition, in the foregoing embodiment in which the sequence of the nucleic acid for ped gene cluster mentioned above may be derived from Pseudomonas putida, for the pedC gene mentioned above, in one specific embodiment, the sequence thereof may comprise, but it is not limited to, the sequence of SEQ ID NO. 4.

Furthermore, in the foregoing embodiment in which the sequence of the nucleic acid for ped gene cluster mentioned above may be derived from Pseudomonas putida, in one specific embodiment, the sequence of the pedA1 gene mentioned above may comprise the sequence of SEQ ID NO. 1, the sequence of the pedA2 gene mentioned above may comprise the sequence of SEQ ID NO. 2, the sequence of the pedB gene mentioned above may comprise the sequence of SEQ ID NO. 3, and the sequence of the pedC gene mentioned above may comprise the sequence of SEQ ID NO. 4.

In addition, in the foregoing embodiment in which the sequence of the nucleic acid for ped gene cluster mentioned above may be derived from Pseudomonas putida, for one specific embodiment, the sequence of the nucleic acid encoding an ATP-binding cassette transporter mentioned above may comprise, but is not limited to, the sequence of SEQ ID NO. 5.

Furthermore, the sequence of the nucleic acid encoding tyrosinase mentioned above in the second exogenous nucleic acid mentioned above may comprise a sequence having more than 85% sequence identity with the sequence of SEQ ID NO. 6, but it is not limited thereto. In one specific embodiment, the sequence of the nucleic acid encoding tyrosinase mentioned above in the second exogenous nucleic acid mentioned above may comprise the sequence of SEQ ID NO. 6.

In one embodiment, the sequence of the nucleic acid encoding tyrosinase mentioned above may be derived from Ralstonia pseudosolanacearum, but it is not limited thereto.

In the foregoing embodiment in which the sequence of the nucleic acid encoding tyrosinase mentioned above may be derived from Ralstonia pseudosolanacearum, in one specific embodiment, the sequence of the nucleic acid encoding tyrosinase mentioned above may comprise the sequence of SEQ ID NO. 6.

The location of the foregoing first exogenous nucleic acid contained in the genetically modified microorganism producing black dyes of the present disclosure has no particular limitation, as long as the foregoing first exogenous nucleic acid can display its intended function, such as encoding a protein that conforms to the sequence information thereof. For example, the foregoing first exogenous nucleic acid contained in the genetically modified microorganism producing black dyes of the present disclosure may be located in an expression vector or may be located in the genome of the genetically modified microorganism producing black dyes of the present disclosure, but it is not limited thereto.

Similarly, the location of the foregoing second exogenous nucleic acid contained in the genetically modified microorganism producing black dyes of the present disclosure also has no particular limitation, as long as the foregoing second exogenous nucleic acid can display its intended function, such as encoding a protein that conforms to the sequence information thereof. For example, the foregoing second exogenous nucleic acid contained in the genetically modified microorganism producing black dyes of the present disclosure also may be located in an expression vector or may be located in the genome of the genetically modified microorganism producing black dyes of the present disclosure, but it is not limited thereto.

In one embodiment, the foregoing first exogenous nucleic acid contained in the genetically modified microorganism producing black dyes of the present disclosure may be located in a first expression vector while the foregoing second exogenous nucleic acid contained in the genetically modified microorganism producing black dyes of the present disclosure may be located in a second expression vector. The first expression vector and the second expression vector mentioned above may independently comprise, a plasmid, a cosmid, a viral vector, a chromosome (artificial chromosome), etc., but they are not limited thereto.

In one specific embodiment, the foregoing first exogenous nucleic acid contained in the genetically modified microorganism producing black dyes of the present disclosure may exist in a first expression vector, and the first expression vector mentioned above is a plasmid, meanwhile, the foregoing second exogenous nucleic acid contained in the genetically modified microorganism producing black dyes of the present disclosure may exist in a second expression vector, and the second expression vector mentioned above is another plasmid.

In addition, the genetically modified microorganism producing black dyes of the present disclosure mentioned above may be a genetically modified strain of Escherichia coli, ITRI-RB3, deposited in Bioresource Collection and Research Centre (BCRC) of Food Industry Research and Development Institute (FIRDI) (ROC.) on Nov. 27, 2023, of which the deposit number is BCRC 940700.

In addition, the present disclosure also provides a method for producing black dyes.

The method for producing black dyes of the present disclosure mentioned above may comprise the following step, but it is not limited thereto.

First, a genetically modified microorganism producing black dyes is cultured in the presence of a rare earth element and/or ion, and the foregoing genetically modified microorganism producing black dyes may be any of the genetically modified microorganism producing black dyes of the present disclosure mentioned above.

The rare earth element and/or ion mentioned above may be any rare earth elements and/or ions, or any combination thereof, and has no particular limitation. In one embodiment, the rare earth element mentioned above may comprise, but is not limited to, at least one of lanthanum, cerium, and erbium. Moreover, in one embodiment, the rare earth ion mentioned above may comprise, but is not limited to, at least one of lanthanum ion, cerium ion, and erbium ion.

In one embodiment, for the method for producing black dyes of the present disclosure, the rare earth element and/or ion mentioned above may be a rare earth ion, and the rare earth ion mentioned above may comprise, but is not limited to, at least one of lanthanum ion, cerium ion, and erbium ion.

For the method for producing black dyes of the present disclosure, in one embodiment, the concentration of the rare earth element and/or ion mentioned above may be about 10-1000 mg/L, such as about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L, about 100 mg/L, about 125 mg/L, about 150 mg/L, about 200 mg/L, about 250 mg/L, about 300 mg/L, about 400 mg/L, about 500 mg/L, about 600 mg/L, about 700 mg/L, about 800 mg/L, about 900 mg/L, about 1000 mg/L, but it is not limited thereto. In one specific embodiment, for the method for producing black dyes of the present disclosure, the rare earth element and/or ion mentioned above may be a rare earth ion, and the rare earth ion mentioned above may comprise, but is not limited to, at least one of lanthanum ion, cerium ion, and erbium ion, and the concentration of the rare earth ion mentioned above may be about 10-1000 mg/L, such as about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L, about 100 mg/L, about 125 mg/L, about 150 mg/L, about 200 mg/L, about 250 mg/L, about 300 mg/L, about 400 mg/L, about 500 mg/L, about 600 mg/L, about 700 mg/L, about 800 mg/L, about 900 mg/L, about 1000 mg/L, but it is not limited thereto.

In the method for producing black dyes of the present disclosure, in one embodiment, the genetically modified microorganism producing black dyes mentioned above may be cultured at about 25-40° C., such as about 25° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is not limited thereto.

Moreover, in the method for producing black dyes of the present disclosure, in one embodiment, the genetically modified microorganism producing black dyes mentioned above may be cultured at about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH 8.0, but it is not limited thereto.

Furthermore, in the method for producing black dyes of the present disclosure, in one embodiment, the genetically modified microorganism producing black dyes mentioned above may be cultured for about 4-24 hours, such as about 4 hours, about 5 hours, about 6 hours, about 7 hours, about 8 hours, about 9 hours, about 10 hours, about 11 hours, about 12 hours, about 15 hours, about 16 hours, about 18 hours, about 21 hours, about 24 hours, but it is not limited thereto.

In addition, in the method for producing black dyes of the present disclosure, a culture medium used to culture the genetically modified microorganism producing black dyes mentioned above has no particular limitation, as long as the genetically modified microorganism producing black dyes mentioned above can grow therein and produce black dyes. In one embodiment, in the method for producing black dyes of the present disclosure, the culture medium used to culture the genetically modified microorganism producing black dyes mentioned above may be Lysogeny broth (Luria Bertani broth) medium, a phosphate buffer (PB) with glucose, etc., but it is not limited thereto. In one specific embodiment, the culture medium used to culture the genetically modified microorganism producing black dyes mentioned above may be Lysogeny broth medium. In another embodiment, the culture medium used to culture the genetically modified microorganism producing black dyes mentioned above may be a phosphate buffer with about 5-15 g/L glucose, such as a phosphate buffer with about 10 g/L glucose.

Moreover, in another embodiment, the method for producing black dyes of the present disclosure may further comprise extracting black dyes from the genetically modified microorganism producing black dyes mentioned above after the step of culturing a genetically modified microorganism producing black dyes in the presence of a rare earth element and/or ion mentioned above is completed, but it is not limited thereto.

The manner for extracting black dyes from the genetically modified microorganism producing black dyes mentioned above has no particular limitation, as long as the black dyes produced by the microorganism can be separated from the microorganism. For example, the microorganism can be cell disrupted first and then centrifuged to obtain the black dyes exist in the supernatant, but it is not limited thereto.

Furthermore, in one specific embodiment, the genetically modified microorganism producing black dyes used in the method for producing black dyes of the present disclosure may be a genetically modified strain of Escherichia coli, ITRI-RB3, deposited in Bioresource Collection and Research Centre (BCRC) of Food Industry Research and Development Institute (FIRDI) (ROC.) on Nov. 27, 2023, of which the deposit number is BCRC 940700.

### EXAMPLES

### A. Material and Methods

### 1. Genetic Modification for Escherichia coli

### (1) Construction of Plasmid V-RE1

The nucleic acid sequences of pedA1 gene, pedA2 gene pedB gene and pedC gene belonging to the ped gene cluster derived from the genome of Pseudomonas putida KT2440 were obtained from the National Center for Biotechnology Information (NCBI) database, wherein the sequence of pedA1 gene is the sequence of SEQ ID NO. 1 (the amino acid sequence encoded thereby is the sequence of SEQ ID NO. 7), the sequence of pedA2 gene is the sequence of SEQ ID NO. 2 (the amino acid sequence encoded thereby is the sequence of SEQ ID NO. 8), the sequence of pedB gene is the sequence of SEQ ID NO. 3 (the amino acid sequence encoded thereby is the sequence of SEQ ID NO. 9), and the sequence of pedC gene is the sequence of SEQ ID NO. 4 (the amino acid sequence encoded thereby is the sequence of SEQ ID NO. 10). The ped gene cluster constituted by pedA1 gene, pedA2 gene, pedB gene and pedC gene can encode an ATP-binding cassette transporter (ABC transporter), PedA1A2BC, which can serve as a rare earth ion channel. More specifically, pedA1 gene, pedA2 gene, pedB gene and pedC gene can respectively encode PedA1 protein (the sequence thereof is the sequence of SEQ ID NO. 7), PedA2 protein (the sequence thereof is the sequence of SEQ ID NO. 8), PedB protein (the sequence thereof is the sequence of SEQ ID NO. 9) and PedC protein (the sequence thereof is the sequence of SEQ ID NO. 10) while PedA1 protein, PedA2 protein, PedB protein and PedC protein can constitute the foregoing ATP-binding cassette transporter.

By sequentially connecting the nucleic acid sequences of the foregoing pedA1 gene, pedA2 gene, pedB gene and pedC gene, a nucleic acid sequence encoding the ATP-binding cassette transporter PedA1A2BC can be obtained (the sequence of SEQ ID NO. 5).

For the nucleic acid sequence encoding the ATP-binding cassette transporter PedA1A2BC (the sequence of SEQ ID NO. 5) mentioned above, primers for polymerase chain reaction (forward primer: TGCGTCAACCAGCTCATCTGGCGCTGACC (SEQ ID NO. 12); reverse primer: TTAACGCGGTTTACGCAGAGCCGCGTGCTG (SEQ ID NO. 13)) and restriction enzyme cutting sites were designed to meet the requirements of multiple cloning sites of plasmid.

The above-mentioned nucleic acid sequence encoding the ATP-binding cassette transporter PedA1A2BC (the sequence thereof was the sequence of SEQ ID NO. 5) was amplified through a polymerase chain reaction using the above-mentioned primers, and then the amplified DNA fragment was purified by electrophoresis. The obtained purified DNA fragment was ligated to plasmid pET-21b via specific restriction enzymes to obtain plasmid V-RE1.

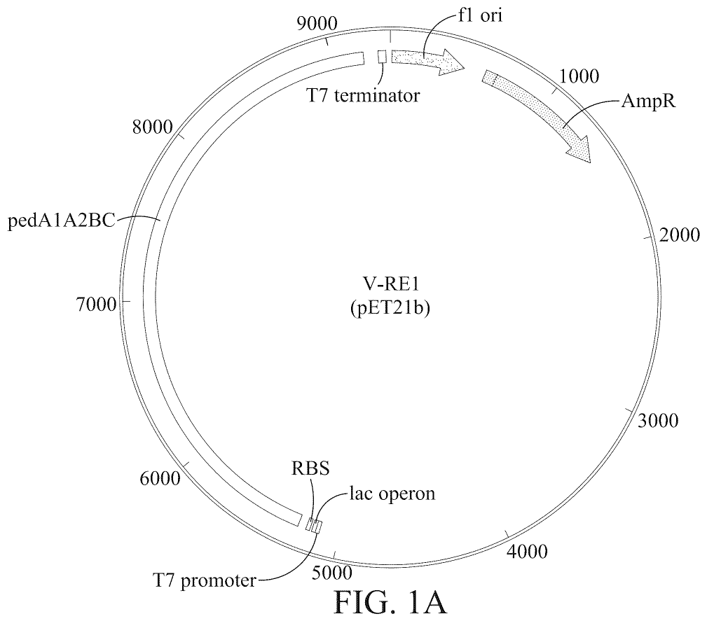

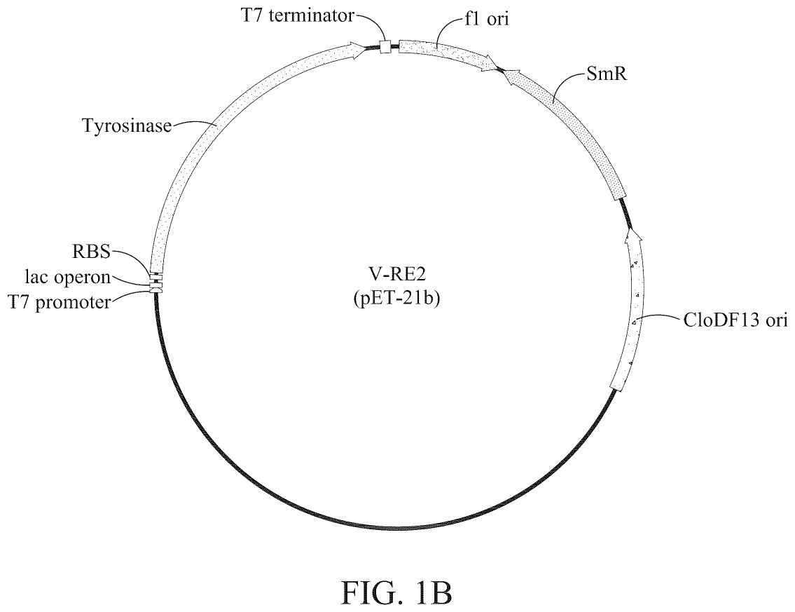

The map of plasmid V-RE1 is shown in FIG. 1A, which carries the nucleic acid encoding the ATP-binding cassette transporter PedA1A2BC (the sequence thereof was the sequence of SEQ ID NO. 5).

### (2) Construction of Plasmid V-RE2

The nucleic acid sequence encoding tyrosinase derived from the genome of Ralstonia pseudosolanacearum was obtained from the National Center for Biotechnology Information (NCBI) database (the sequence of SEQ ID NO. 6, the amino acid sequence encoded thereby is the sequence of SEQ ID NO. 11).

For the foregoing nucleic acid sequence encoding tyrosinase (the sequence of SEQ ID NO. 6), primers for polymerase chain reaction (forward primer: ATGGTAGTCCGTCGTACCGTGCTTAAGGCG (SEQ ID NO. 14)); reverse primer: TTAAATCACTGCCACCTCAATGCTTTCCGG (SEQ ID NO.15)) and restriction enzyme cutting sites were designed to meet the requirements of multiple cloning sites of plasmid.

The foregoing nucleic acid sequence encoding tyrosinase (the sequence thereof was the sequence of SEQ ID NO. 6) was amplified through a polymerase chain reaction using the above-mentioned primers, and then the amplified DNA fragment was purified by electrophoresis. The obtained purified DNA fragment was ligated to plasmid pET-21b via specific restriction enzymes to obtain plasmid V-RE2.

The map of plasmid V-RE2 is shown in FIG. 1B, which carries the nucleic acid sequence encoding tyrosinase (the sequence thereof was the sequence of SEQ ID NO. 6).

(3) Construction of Genetically Modified Strains of Escherichia coli, ITRI-RB1, ITRI-RB2 and ITRI-RB3

The plasmid V-REI and/or plasmid V-RE2 obtained above was/were introduced into competent cells of Escherichia coli BL-21, and the antibiotic(s) corresponding to the plasmid(s) was/were used for tolerance screening. After the obtained strain was confirmed that whether the length of the gene fragment which has been introduced matches the length of the gene fragment to be introduced through a polymerase chain reaction, gene sequencing was performed to confirm that the gene fragment to be introduced has indeed been introduced into Escherichia coli BL-21.

Genetically modified strains of Escherichia coli, ITRI-RB1, ITRI-RB2 and ITRI-RB3 were constructed through the method mentioned above. Genetically modified strain of Escherichia coli, ITRI-RB1, contains plasmid V-REI, genetically modified strain of Escherichia coli, ITRI-RB2, contains plasmid V-RE2 (having capacity to produce black dyes), and genetically modified strain of Escherichia coli, ITRI-RB3, contains plasmid V-RE1 and plasmid V-RE2 at the same time (having capacity to produce black dyes).

Subsequently, the growth ability and/or the capacity to produce black dyes of genetically modified strains of Escherichia coli, ITRI-RB1, ITRI-RB2 and ITRI-RB3 were determined in the presence or absence of rare earth ions.

### 2. Medium Formula

Medium A: Lysogeny broth (Luria Bertani broth) medium (which is a nutritional medium commonly used for microbial culture).
Medium B: Glucose 10 g/L, phosphate buffer (PB), and addition of antibiotics, ampicillin (100 mg/mL) or spectinomycin (40 mg/mL) or addition of both two antibiotics at the same time according to the difference of test strains.

### 3. Analytical Method for Black Dye Production of Bacterial Strains

For quantitative analysis of black dye production, please refer to the literature: “Zhichao Wu et al; Production, physico-chemical characterization and antioxidant activity of natural melanin from submerged cultures of the mushroom Auricularia auricular, 2018; Food Bioscience”.

### (1) Establishment of Standard Curve

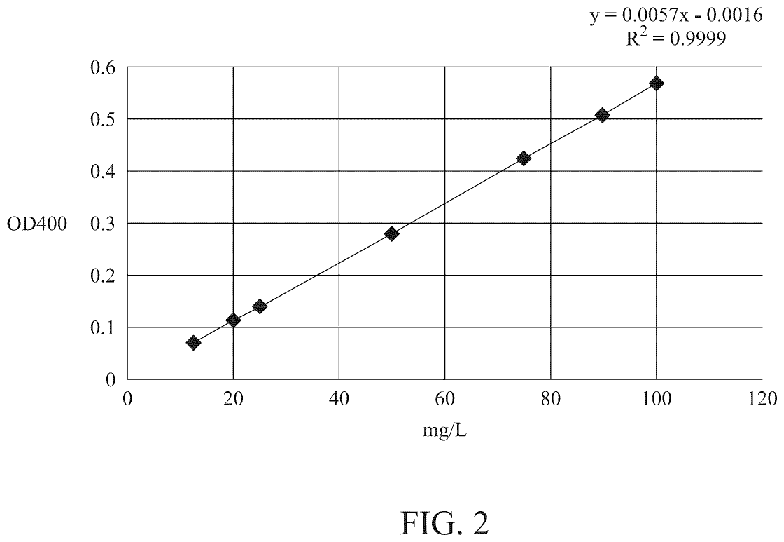

The black dye (melanin) standard was prepared to standard samples with different concentrations using 0.1N NaOH. Absorbance values (OD400) of standard samples with different concentrations were measured at a wavelength of 400 nm by a spectrophotometer (Table 1), and a standard curve of black dye concentration versus OD400 was established (FIG. 2).

TABLE 1
Absorbance values measured at wavelength 400
nm for various concentrations of black dye

Absorbance value of blank (NaOH)
0.046

| | | Absorbance value |
|---|---|---|
| | | (absorbance value of |
| Black | Absorbance | the blank group has |
| dye (mg/L) | value | been deducted) |
| 200 | 1.187 | 1.141 |
| 100 | 0.614 | 0.568 |
| 90 | 0.552 | 0.506 |
| 75 | 0.470 | 0.424 |
| 50 | 0.324 | 0.278 |
| 25 | 0.185 | 0.139 |
| 20 | 0.159 | 0.113 |
| 12.5 | 0.117 | 0.071 |
| 6.25 | 0.082 | 0.036 |

### (2) Determination of Black Dye Production of Bacterial Strains

After the culture was completed, the culture medium containing bacteria was centrifuged, and then the supernatant was removed. 0.01-0.5 N NaOH was added to the bacterial cells, and then cell disruption was conducted by ultrasonic vibration to obtain a cell lysate solution.

Next, the cell lysate solution was centrifuged, and the absorbance value of the obtained supernatant was measured at a wavelength of 400 nm. After that, the measured absorbance value was used to calculate the black dye content in the supernatant according to the standard curve of black dye concentration versus OD400 mentioned above to confirm the black dye production of the bacteria.

### B. Experiments

### Example 1

### Effect of Increasing the Concentration of Rare Earth Ions (Lanthanum Ions (La³⁺)) on the Growth of the Modified Strain of Escherichia coli, ITRI-RB1

The modified strain of Escherichia coli, ITRI-RB1, was inoculated into a 14 mL culture tube containing 2 mL of Medium A and cultured overnight at 37° C. to activate the strain. After that, the modified strain of Escherichia coli, ITRI-RB1, was inoculated into shake flasks containing Medium B, 0.5 mM isopropyl β-D-1-thiogalactopyranoside (IPTG) and different concentrations of rare earth ions (lanthanum ions (La³⁺)) (0 mg/L, 50 mg/L, 100 mg/L, 250 mg/L and 500 mg/L) and cultured at 30° C., pH 7.0.

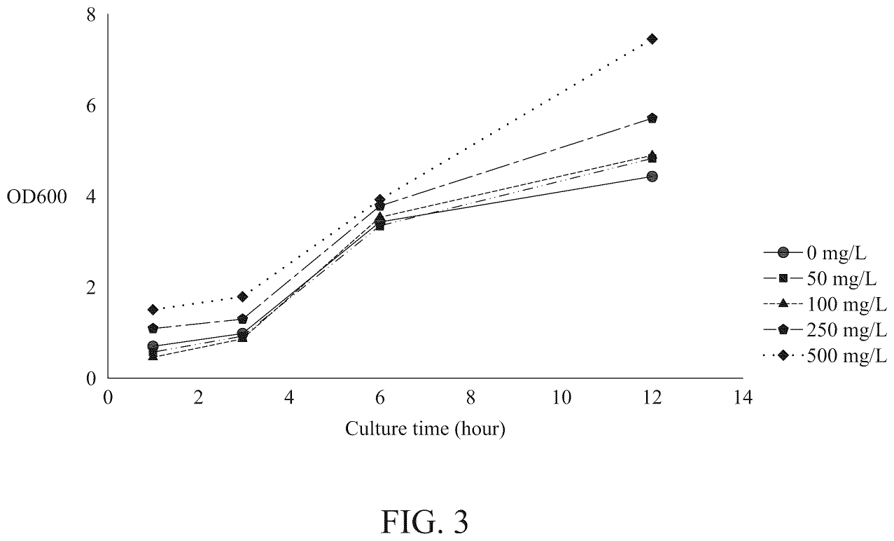

During the culture period, the bacterial cell suspension was taken out at different time points, and the absorbance value (OD600) of the bacterial cell suspension at a wavelength of 600 nm was measured with a spectrophotometer to analyze the growth ability of the modified strain of Escherichia coli, ITRI-RB1. The results are shown in FIG. 3.

FIG. 3 shows that as the concentration of rare earth ions (lanthanum ions (La³⁺)) increases, the growth ability (OD600) of the modified strain of Escherichia coli, ITRI-RB1, also increases. At 12 hours of culture, compared to culturing in the absence of rare earth ions, the growth ability of the modified strain of Escherichia coli, ITRI-RB1, was increased by approximately 68% when cultured in the presence of 500 mg/L rare earth ions (lanthanum ions (La³⁺)).

According to the foregoing, it is understood that the growth of bacterial strain with rare earth ion channels (ATP-binding cassette transporter PedA1A2BC) can be promoted by addition of rare earth ions.

### Example 2

### Improvement Effect of Different Rare Earth Ions on the Growth Ability of the Modified Strain of Escherichia coli, ITRI-RB1

The modified strain of Escherichia coli, ITRI-RB1, was inoculated into a 14 mL culture tube containing 2 mL of Medium A and cultured overnight at 37° C. to activate the strain. After that, the modified strain of Escherichia coli, ITRI-RB1, was inoculated into shake flasks containing Medium B, 0.5 mM isopropyl β-D-1-thiogalactopyranoside (IPTG) and different rare earth ions (0 mg/L, or 500 mg/L lanthanum ions (La³⁺), cerium ions (Ce³⁺) or erbium ions (Er³⁺)) and cultured at 30° C., pH 7.0.

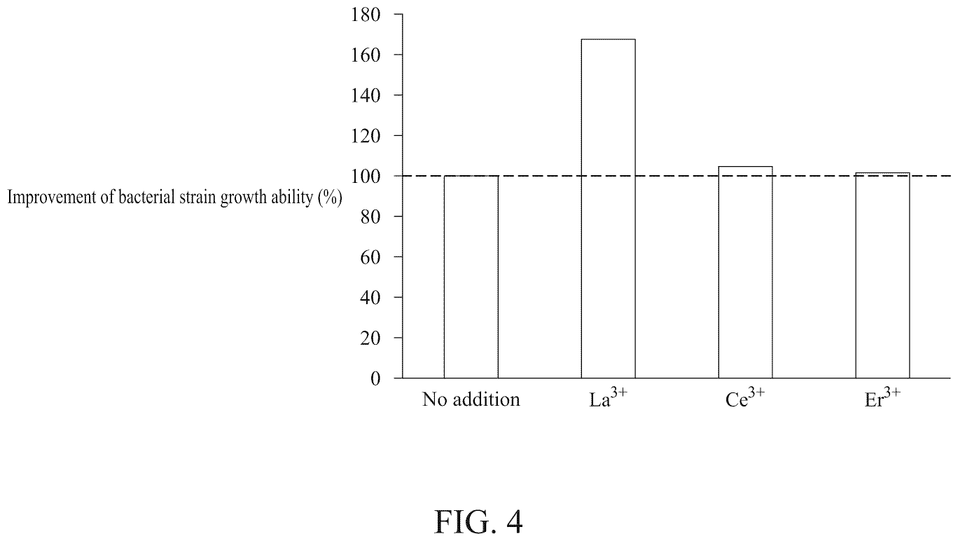

After 12 hours of culture, the bacterial cell suspension was taken out, and the absorbance value (OD600) of the bacterial cell suspension at a wavelength of 600 nm was measured with a spectrophotometer to analyze the effects of different rare earth ions on the growth ability of the modified strain of Escherichia coli, ITRI-RB1. The results are shown in FIG. 4.

FIG. 4 shows that different rare earth ions can improve the growth ability of the modified strain of Escherichia coli, ITRI-RB1, wherein compared to slight improvement on bacterial growth by cerium ions (Ce³⁺) and erbium ions (Er³⁺), lanthanum ions (La³⁺) have a better improvement effect on bacterial growth ability.

According to the foregoing, it is understood that for the bacterial strain with rare earth ion channels, addition of lanthanum ions (La³⁺) can greatly improve the growth ability of the bacterial strain.

### Example 3

### Rare Earth Ion Uptake of Escherichia coli BL21 and the Modified Strain of Escherichia coli, ITRI-RB1

Escherichia coli BL21 (no plasmid introduction) and the modified strain of Escherichia coli, ITRI-RB1, were individually inoculated into a 14 mL culture tube containing 2 mL of Medium A and cultured overnight at 37° C. to activate the strains. After that, Escherichia coli BL21 (no plasmid introduction) and the modified strain of Escherichia coli, ITRI-RB1, were individually inoculated into a shake flask containing Medium B, 0.5 mM isopropyl β-D-1-thiogalactopyranoside (IPTG) and different concentrations of rare earth ions (250 mg/L or 500 mg/L lanthanum ions (La³⁺)) and cultured at 30° C., pH 7.0.

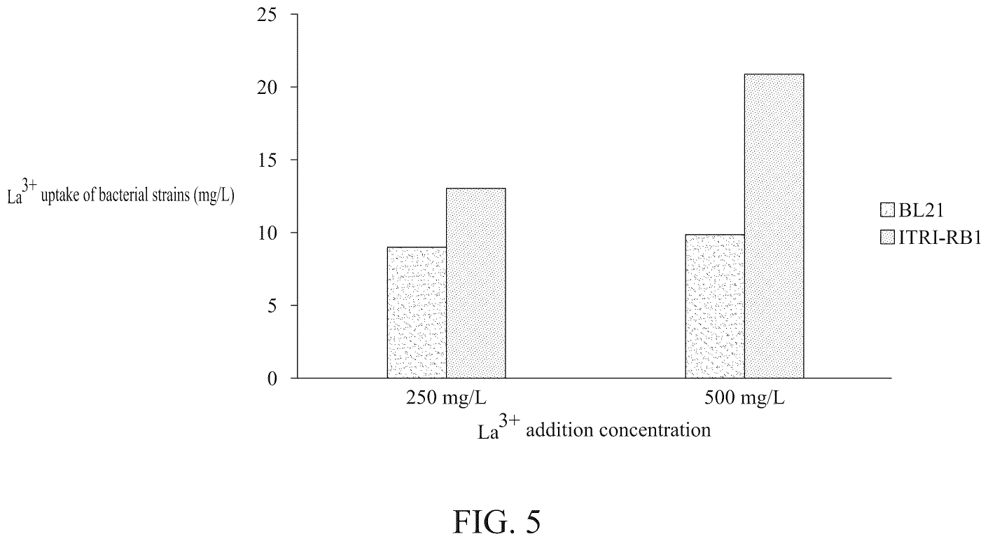

After 12 hours of culture, the bacterial cell suspensions were taken out and centrifuged to obtain supernatants. The obtained supernatants were analyzed for lanthanum ion (La³⁺) concentration using an inductively coupled plasma optical emission spectrometry (ICP-OES), and the rare earth ion uptakes of Escherichia coli BL21 and the modified strain of Escherichia coli, ITRI-RB1, were obtained thereby. The results are shown in FIG. 5.

FIG. 5 shows that the modified strain of Escherichia coli, ITRI-RB1, has better rare earth ion uptake efficiency than its source bacterial strain (E. coli BL21 (no plasmid introduction)).

According to the foregoing, it is understood that for the bacterial strain with rare earth ion channels, rare earth ion uptake efficiency thereof is improved.

### Example 4

### Effect of Rare Earth Ions (Lanthanum Ions (La³⁺)) on the Growth of the Modified Strains of Escherichia coli, ITRI-RB2 and ITRI-RB3

The modified strain of Escherichia coli, ITRI-RB2 and the modified strain of Escherichia coli, ITRI-RB3, were individually inoculated into a 14 mL culture tube containing 2 mL of Medium A and cultured overnight at 37° C. to activate the strains. After that, the modified strain of Escherichia coli, ITRI-RB2 and the modified strain of Escherichia coli, ITRI-RB3, were individually inoculated into a shake flask containing Medium B, 0.5 mM isopropyl β-D-1-thiogalactopyranoside (IPTG) and rare earth ions (500 mg/L lanthanum ions (La3+)) and cultured at 30° C., pH 7.0.

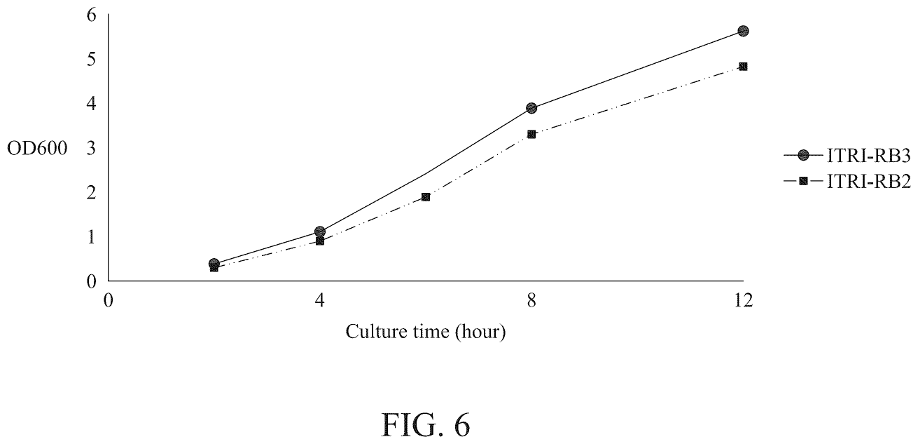

During the culture period, the bacterial cell suspensions were taken out at different time points, and the absorbance values (OD600) of the bacterial cell suspensions at a wavelength of 600 nm were measured with a spectrophotometer to analyze the growth ability of the modified strains of Escherichia coli, ITRI-RB2 and ITRI-RB3. The results are shown in FIG. 6.

FIG. 6 shows that in the presence of rare earth ions, the modified strain of Escherichia coli, ITRI-RB3, which has two plasmids, plasmid V-RE1 and plasmid V-RE2, at the same time has better growth ability than the modified strain of Escherichia coli, ITRI-RB2, which has only one plasmid, plasmid V-RE2.

According to the foregoing, it is understood that in the presence of rare earth ions, for black dye-producing bacterial strains, having rare earth ion channels can improve growth ability.

### Example 5

The modified strain of Escherichia coli, ITRI-RB2 and the modified strain of Escherichia coli, ITRI-RB3, were individually inoculated into a 14 mL culture tube containing 2 mL of Medium A and cultured overnight at 37° C. to activate the strains. After that, the modified strain of Escherichia coli, ITRI-RB2 and the modified strain of Escherichia coli, ITRI-RB3, were individually inoculated into a shake flask containing Medium B, 5 g/L tyrosine, 0.5 mM isopropyl β-D-1-thiogalactopyranoside (IPTG) and rare earth ions (500 mg/L lanthanum ions (La³⁺)) and cultured at 30° C., pH 7.0.

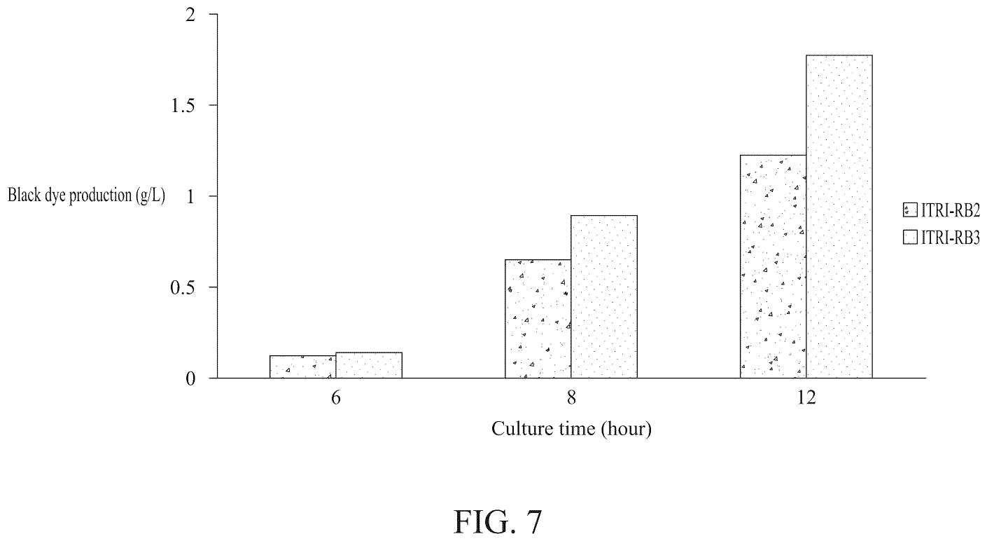

At 6, 8 and 12 hours of culture, the bacterial cell suspension was taken out and black dye production analysis was performed. The results are shown in FIG. 7.

FIG. 7 shows that in the presence of rare earth ions, the modified strain of Escherichia coli, ITRI-RB3, which has two plasmids, plasmid V-REI and plasmid V-RE2, at the same time has better black dye production capacity than the modified strain of Escherichia coli, ITRI-RB2, which has only one plasmid, plasmid V-RE2.

According to the foregoing, it is understood that in the presence of rare earth ions, for black dye-producing bacterial strains, having rare earth ion channels can improve black dye production capacity.

In addition, the modified strain of Escherichia coli, ITRI-RB3, was deposited in Bioresource Collection and Research Centre (BCRC) of Food Industry Research and Development Institute (FIRDI) (ROC.) on Nov. 27, 2023 with the deposit number of BCRC 940700.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A genetically modified microorganism producing black dyes, comprising:
a first exogenous nucleic acid, which comprises a nucleic acid encoding an ATP-binding cassette transporter (ABC transporter), wherein the nucleic acid encoding an ATP-binding cassette transporter comprises a nucleic acid for ped gene cluster; and
a second exogenous nucleic acid, which comprises a nucleic acid encoding tyrosinase.

2. The genetically modified microorganism producing black dyes as claimed in claim 1, wherein a source microorganism of the genetically modified microorganism producing black dyes comprises a bacterium, an actinomycete, a yeast or a mold, wherein the bacterium comprises Escherichia coli, Corynebacterium glutamicum or Bacillus subtilis, and the yeast comprises Yarrowia lipolytica, Saccharomyces cerevisiae or Pichia pastoris.

3. The genetically modified microorganism producing black dyes as claimed in claim 1, wherein the ped gene cluster comprises pedA1 gene, pedA2 gene, pedB gene and pedC gene, wherein the sequence of the pedA1 gene comprises a sequence having more than 85% sequence identity with the sequence of SEQ ID NO. 1, the sequence of the pedA2 gene comprises a sequence having more than 85% sequence identity with the sequence of SEQ ID NO. 2, the sequence of the pedB gene comprises a sequence having more than 85% sequence identity with the sequence of SEQ ID NO. 3 and the sequence of the pedC gene comprises a sequence having more than 85% sequence identity with the sequence of SEQ ID NO. 4, and wherein the sequence of the nucleic acid encoding an ATP-binding cassette transporter comprises a sequence having more than 85% sequence identity with the sequence of SEQ ID NO. 5.

4. The genetically modified microorganism producing black dyes as claimed in claim 3, wherein the ped gene cluster is derived from Pseudomonas putida.

5. The genetically modified microorganism producing black dyes as claimed in claim 4, wherein the sequence of the pedA1 gene comprises the sequence of SEQ ID NO. 1, the sequence of the pedA2 gene comprises the sequence of SEQ ID NO. 2, the sequence of the pedB gene comprises the sequence of SEQ ID NO. 3, and the sequence of the pedC gene comprises the sequence of SEQ ID NO. 4.

6. The genetically modified microorganism producing black dyes as claimed in claim 4, wherein the sequence of the nucleic acid encoding an ATP-binding cassette transporter comprises the sequence of SEQ ID NO. 5.

7. The genetically modified microorganism producing black dyes as claimed in claim 1, wherein the sequence of the nucleic acid encoding tyrosinase comprises a sequence having more than 85% sequence identity with the sequence of SEQ ID NO. 6.

8. The genetically modified microorganism producing black dyes as claimed in claim 1, wherein the sequence of the nucleic acid encoding tyrosinase is derived from Ralstonia pseudosolanacearum.

9. The genetically modified microorganism producing black dyes as claimed in claim 8, wherein the sequence of the nucleic acid encoding tyrosinase comprises the sequence of SEQ ID NO. 6.

10. The genetically modified microorganism producing black dyes as claimed in claim 1, wherein the first exogenous nucleic acid is located in a first expression vector and the second exogenous nucleic acid is located in a second expression vector, and wherein the first expression vector and the second expression vector independently comprises a plasmid, a cosmid, a viral vector or a chromosome.

11. The genetically modified microorganism producing black dyes as claimed in claim 1, wherein the genetically modified microorganism producing black dyes is a genetically modified strain of Escherichia coli of which the deposit number is BCRC 940700.

12. A method for producing black dyes, comprising:
(a) culturing a genetically modified microorganism producing black dyes in the presence of a rare earth element and/or ion,
wherein the genetically modified microorganism producing black dyes comprises:
a first exogenous nucleic acid, which comprises a nucleic acid encoding an ATP-binding cassette transporter (ABC transporter), wherein the nucleic acid encoding an ATP-binding cassette transporter comprises a nucleic acid for ped gene cluster; and
a second exogenous nucleic acid, which comprises a nucleic acid encoding tyrosinase.

13. The method for producing black dyes as claimed in claim 12, wherein a source microorganism of the genetically modified microorganism producing black dyes comprises a bacterium, an actinomycete, a yeast or a mold, wherein the bacterium comprises Escherichia coli, Corynebacterium glutamicum or Bacillus subtilis, and the yeast comprises Yarrowia lipolytica, Saccharomyces cerevisiae or Pichia pastoris.

14. The method for producing black dyes as claimed in claim 12, wherein the ped gene cluster comprises pedA1 gene, pedA2 gene, pedB gene and pedC gene.

15. The method for producing black dyes as claimed in claim 14, wherein the ped gene cluster is derived from Pseudomonas putida, and wherein the sequence of the pedA1 gene comprises the sequence of SEQ ID NO. 1, the sequence of the pedA2 gene comprises the sequence of SEQ ID NO. 2, the sequence of the pedB gene comprises the sequence of SEQ ID NO. 3, and the sequence of the pedC gene comprises the sequence of SEQ ID NO. 4.

16. The method for producing black dyes as claimed in claim 15, wherein the sequence of the nucleic acid encoding an ATP-binding cassette transporter comprises the sequence of SEQ ID NO. 5.

17. The method for producing black dyes as claimed in claim 12, wherein the sequence of the nucleic acid encoding tyrosinase is derived from Ralstonia pseudosolanacearum, and the sequence of the nucleic acid encoding tyrosinase comprises the sequence of SEQ ID NO. 6.

18. The method for producing black dyes as claimed in claim 12, wherein a concentration of the rare earth element and/or ion is 50-1000 mg/L, the genetically modified microorganism producing black dyes is cultured at 25-40° C., the genetically modified microorganism producing black dyes is cultured at pH 6.0-8.0, and the genetically modified microorganism producing black dyes is cultured for 4-24 hours.

19. The method for producing black dyes as claimed in claim 12, further comprising (b) extracting black dyes from the genetically modified microorganism producing black dyes after the step (a) is completed.

20. The method for producing black dyes as claimed in claim 12, wherein the genetically modified microorganism producing black dyes is a genetically modified strain of Escherichia coli of which the deposit number is BCRC 940700.
